(19) United States
(12) Patent Application Publication
(10) Pub. No.: US 2025/0206846 A1
(43) Pub. Date: Jun. 26, 2025

(54) ANTI-KLK4 ANTIBODIES AND USES THEREOF

(71) Applicant: Yeda Research and Development Co. Ltd., Rehovot (IL)

(72) Inventors: Irit SAGI, Rehovot (IL); Nikolaos A. AFRATIS, Rehovot (IL); Ashley M. BUCKLE, Rehovot (IL)

(73) Assignee: Yeda Research and Development Co. Ltd., Rehovot (IL)

(21) Appl. No.: 19/075,900

(22) Filed: Mar. 11, 2025

Related U.S. Application Data
(63) Continuation of application No. PCT/IL2023/050977, filed on Sep. 11, 2023.
(60) Provisional application No. 63/405,446, filed on Sep. 11, 2022.

(51) Int. Cl. C07K 16/40; C12N 9/64; G01N 33/573

(52) U.S. Cl. CPC C07K 16/40; C12N 9/6445; G01N 33/573; C07K 2317/56; C07K 2317/76; C07K 2317/92; G01N 2333/96455

(57) ABSTRACT

An isolated antibody that binds to a cyclized peptide having an amino acid sequence as set forth in SEQ ID NO: 9 with an EC50 of less than 500 nM, as determined by ELISA is disclosed. Uses of same are also disclosed as well as methods of generating same.

### RELATED APPLICATIONS

This application is a Continuation of PCT Patent Application No. PCT/IL2023/050977 having International filing date Sep. 11, 2023, which claims the benefit of priority under 35 USC § 119 (e) of U.S. Provisional Patent Application No. 63/405,446 filed on Sep. 11, 2022. The contents of the above applications are all incorporated by reference as if fully set forth herein in their entirety.

### SEQUENCE LISTING STATEMENT

The XML file, entitled 102928SequenceListing.xml, created on Feb. 10, 2025, comprising 14,779 bytes, submitted concurrently with the filing of this application is incorporated herein by reference.

### FIELD AND BACKGROUND OF THE INVENTION

The present invention, in some embodiments thereof, relates to antibodies that bind KLK4 and uses thereof.

The kallikrein (KLK) gene family is the largest protease gene cluster in the human genome and encodes 15 serine proteases that have sequence identity varying from 40-80%, with a high degree of structural similarity around the active site. The KLK proteases are involved in development and normal physiology but have also been implicated in cancer progression. KLK4 is predominantly expressed in basal and secretory cells of the prostate gland with lower levels of expression in a number of tissues including breast, ovaries, thyroid, testis and developing teeth. Over-expression of KLK4 has been documented in malignant prostate, ovarian and breast tumors and is associated with metastasis, and mechanisms underpinning resistance to androgen deprivation therapy. Conversely, KLK4 inhibition has resulted in reduced proliferation and spheroid formation in tissue culture based systems.

The most direct way to inhibit KLK4 is by designing specific active site inhibitors. Allostery offers another route to KLK4 inhibition. KLK4, like the majority of serine proteases, is tightly regulated by conformational switches. Serine proteases are synthesized in an inactive zymogen state that has a distorted active site unable to efficiently support catalysis. Upon cleavage of the propeptide, the new N-terminal isoleucine/leucine forms a salt bridge with Asp194 (chymotrypsin numbering used throughout) to rigidify the oxyanion hole and active site catalytic triad. While most proteases are fully activated upon cleavage of the zymogen, some proteases use additional conformational switches such as ions or protein binding partners that also alter the rigidity of the active site.

The X-ray crystal structures of KLK4 in complex with both sunflower trypsin inhibitor-1 (SFTI-1) and a rationally SFTI-1 derivative, has been determined to atomic (˜1 Å) resolution (Riley et al, 2016, Sci Rep 6, 35385). It was found that KLK4 loop 3 is allosterically connected via a metal ion to H25, which in turn influences the removal of the N-terminal strand from a functional position within the protease, thus inhibiting the enzyme. This site is structurally and chemically diverse within the Kallikrein family and is thus a unique target for the development of binding molecules with therapeutic potential.

Additional Background Art includes U.S. patent application No. 20210301032, International Patent Application No. WO2011092700A1 and International Patent Application No. WO2021/055577.

### SUMMARY OF THE INVENTION

According to an aspect of some embodiments of the present invention there is provided an isolated antibody that binds to a cyclized peptide having an amino acid sequence as set forth in SEQ ID NO: 9 with an EC50 of less than 500 nM, as determined by ELISA.

According to an aspect of some embodiments of the present invention there is provided an isolated antibody which binds specifically to human KLK4, comprising an antigen recognition domain having complementarity determining region (CDR) amino acid sequences as set forth in: SEQ ID NOs: 1 (CDR1), 2 (CDR2) and 2 (CDR3), sequentially arranged from N to C on a light chain of the antibody) and SEQ ID NOs: 4 (CDR1), 5 (CDR2) and 6 (CDR3), sequentially arranged from N to Con a heavy chain of the antibody.

According to an aspect of some embodiments of the present invention there is provided an isolated antibody that competes for binding with the antibody described herein.

According to an aspect of some embodiments of the present invention there is provided an isolated antibody that binds to the same epitope as the antibody described herein.

According to an aspect of some embodiments of the present invention there is provided an isolated nucleic acid encoding the antibody described herein.

According to an aspect of some embodiments of the present invention there is provided an host cell comprising the nucleic acid described herein.

According to an aspect of some embodiments of the present invention there is provided an method of producing an antibody comprising culturing the host cell described herein so that the antibody is produced.

According to an aspect of some embodiments of the present invention there is provided an immunoconjugate comprising the antibody described herein.

According to an aspect of some embodiments of the present invention there is provided an cyclized peptide comprising the amino acid sequence as set forth in SEQ ID NO: 11.

According to an aspect of some embodiments of the present invention there is provided an pharmaceutical composition comprising the antibody described herein and a pharmaceutically acceptable carrier.

According to an aspect of some embodiments of the present invention there is provided an method of treating a disease associated with an up-regulation of KLK4 in a subject in need thereof comprising administering to the subject a therapeutically effective amount of the antibody described herein, thereby treating the disease.

According to an aspect of some embodiments of the present invention there is provided an method of inhibiting a biological activity of KLK4 comprising contacting cells expressing KLK4 with an effective amount of the antibody described herein, thereby inhibiting the biological activity of KLK4.

According to an aspect of some embodiments of the present invention there is provided an method of screening for an antibody that binds KLK4 comprising contacting a candidate antibody with the cyclized peptide described herein, wherein a binding of the candidate antibody to the cyclized peptide with an affinity above a predetermined threshold is indicative of an antibody that binds to KLK4.

According to some embodiments of the invention, the isolated antibody comprises an antigen recognition domain having complementarity determining region (CDR) amino acid sequences as set forth in: SEQ ID NOs: 1 (CDR1), 2 (CDR2) and 2 (CDR3), sequentially arranged from N to Con a light chain of the antibody) and SEQ ID NOs: 4 (CDR1), 5 (CDR2) and 6 (CDR3), sequentially arranged from N to C on a heavy chain of the antibody.

According to some embodiments of the invention, the isolated antibody is capable of binding to human KLK4 with an EC50 of less than 500 nM, as measured by ELISA.

According to some embodiments of the invention, the light chain comprises an amino acid sequence at least 90% identical to SEQ ID NO: 7.

According to some embodiments of the invention, the heavy chain comprises an amino acid sequence at least 90% identical to SEQ ID NO: 8.

According to some embodiments of the invention, the isolated antibody is capable of binding the human KLK4 with a higher affinity as compared to human KLK10, as measured by

ELISA.

According to some embodiments of the invention, the antibody is capable of inhibiting KLK4 protease activity with an IC50 of less than 100 nM.

According to some embodiments of the invention, the isolated antibody is a monoclonal antibody.

According to some embodiments of the invention, the antibody is a monospecific antibody.

According to some embodiments of the invention, the antibody is a bi-specific antibody.

According to some embodiments of the invention, the isolated antibody is for use in treating a disease associated with an up-regulation of KLK4.

According to some embodiments of the invention, the cyclized peptide consists of the amino acid sequence as set forth in SEQ ID NO: 9.

According to some embodiments of the invention, the cyclized peptide is coupled to an antigenically neutral carrier.

According to some embodiments of the invention, the antigenically neutral carrier comprises keyhole limpet hemocyanin (KLH) or serum albumin.

According to some embodiments of the invention, the disease is cancer.

According to some embodiments of the invention, the cancer is selected from the group consisting of prostate, breast and ovarian cancer.

According to some embodiments of the invention, the disease is an inflammatory skin disease.

According to some embodiments of the invention, the inflammatory skin disease is selected from the group consisting of Netherton Syndrome, atopic dermatitis and psoriasis.

According to some embodiments of the invention, the contacting is effected in vivo.

According to some embodiments of the invention, the contacting is effected ex vivo.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B: Cartoon illustrating a cyclized peptide (FIG. 1B) used to mimic the loop 3 allosteric site of KLK4 (FIG. 1A).

FIGS. 2A-B: Binding interaction of anti-KLK4 antibody with recombinant human KLK4 (FIG. 2A) and synthetic peptide (loop-KLK4) (FIG. 2B).

FIG. 3 is a photograph of a gel illustrating inhibition of kallikrein proteolysis of fibrinogen by anti-KLK4 antibody.

FIG. 4 illustrates the effect of the anti-KLK4 antibody on migration of ES2 ovarian cancer cells.

FIG. 5 illustrates the effect of the anti-KLK4 antibody on migration of OVCAR3 ovarian cancer cells

FIGS. 6A-B illustrates the effect of the anti-KLK4 antibody on proliferation of ES2 (FIG. 6A) and OVCAR3 (FIG. 6B) ovarian cancer cells.

FIG. 7 is a model of the KLK4-Antibody complex. Cartoon of the Fv portion of the antibody is shown (heavy chain=cyan, light chain=magenta). KLK4 represented as a semi-transparent molecular surface, with Loop 3 colored orange. CDR3 of heavy chain is labelled (H3).

### DESCRIPTION OF SPECIFIC EMBODIMENTS OF THE INVENTION

The present invention, in some embodiments thereof, relates to antibodies that bind KLK4 and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Members of the Kallikrein family of serine proteases are implicated in the development and metastasis of a wide range of cancers as well as inflammatory skin diseases such as Netherton Syndrome, atopic dermatitis and psoriasis.

However, high sequence and structural conservation at the kallikrein active site presents challenges for the development of specific inhibitors of individual members of the family.

The present inventor previously identified a mechanism of allosteric inhibition in Kallikrein-4 (KLK4; Riley et al., 2016) involving loop 3 of the enzyme. KLK4 loop 3 was found to be allosterically connected via a metal ion to H25, which in turn influences the removal of the N-terminal strand from a functional position within the protease, thus inhibiting the enzyme.

Whilst conceiving embodiments of the present invention, the present inventors have synthesized a cyclized peptide having a sequence and 3D structure that mimics the KLK4 allosteric site. Injection of this peptide into mice, together with classical adjuvants resulted in the generation of antibodies which specifically target KLK4.

Thus, according to an aspect of the invention there is provided an isolated antibody that binds to a cyclized peptide having an amino acid sequence as set forth in SEQ ID NO: 9 with an EC50 of less than 500 nM, as determined by ELISA.

As used herein the term “antibody”, encompasses various antibody structures, including but not limited to monoclonal antibodies, polyclonal antibodies, multispecific antibodies (e.g., bispecific antibodies), and antibody fragments so long as they exhibit the desired antigen-binding activity.

According to specific embodiments the antibody is a recombinant antibody.

As used herein, the term “recombinant antibody” refers an antibody produced by recombinant DNA techniques, i.e., produced from host cells transformed by an exogenous DNA construct encoding the antibody. Exemplary host cells include, but are not limited to

According to another embodiment, the antibody is a monoclonal antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The phrase “antibody fragment” refers to a functional fragment thereof, such as Fab, F(ab′)₂, and Fv that are capable of binding to macrophages. These functional antibody fragments are defined as follows: (i) Fab, the fragment which contains a monovalent antigen-binding fragment of an antibody molecule, can be produced by digestion of whole antibody with the enzyme papain to yield an intact light chain and a portion of one heavy chain; (ii) Fab′, the fragment of an antibody molecule that can be obtained by treating whole antibody with pepsin, followed by reduction, to yield an intact light chain and a portion of the heavy chain; two Fab′ fragments are obtained per antibody molecule; (iii) (Fab′)₂, the fragment of the antibody that can be obtained by treating whole antibody with the enzyme pepsin without subsequent reduction; F(ab′)₂ is a dimer of two Fab′ fragments held together by two disulfide bonds; (iv) Fv, defined as a genetically engineered fragment containing the variable region of the light chain and the variable region of the heavy chain expressed as two chains; (v) Single chain antibody (“SCA”), a genetically engineered molecule containing the variable region of the light chain and the variable region of the heavy chain, linked by a suitable polypeptide linker as a genetically fused single chain molecule; and (vi) Peptides coding for a single complementarity-determining region (CDR).

The “class” of an antibody refers to the type of constant domain or constant region possessed by its heavy chain. There are five major classes of antibodies: IgA, IgD, IgE. IgG, and IgM, and several of these may be further divided into subclasses (isotypes), e.g., IgGi, IgG2, IgG3, IgG4, IgAi, and IgA2. In certain aspects, the antibody is of the IgGi isotype. In certain aspects, the antibody is of the IgGi isotype. In other aspects, the antibody is of the IgG2 isotype. In certain aspects, the antibody is of the IgG4 isotype. The heavy chain constant domains that correspond to the different classes of immunoglobulins are called a, d, e, g, and m, respectively. The light chain of an antibody may be assigned to one of two types, called kappa (κ) and lambda (1), based on the amino acid sequence of its constant domain.

Antibody fragments can be obtained using methods well known in the art. (See for example, Harlow and Lane, Antibodies: A Laboratory Manual, Cold Spring Harbor Laboratory, New York, 1988, incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Alternatively, antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73:119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2:97-105, 1991; Bird et al., Science 242:423-426, 1988; Pack et al., Bio/Technology 11:1271-77, 1993; and Ladner et al., U.S. Pat. No. 4,946,778.

CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2:106-10, 1991.

Methods of generating antibodies (i.e., monoclonal and polyclonal) are well known in the art. Antibodies may be generated via any one of several methods known in the art, which methods can employ induction of in vivo production of antibody molecules, screening immunoglobulin libraries or panels of highly specific binding reagents as disclosed [Orlandi D. R. et al. (1989) Proc. Natl. Acad. Sci. 86:3833-3837, Winter G. et al. (1991) Nature 349:293-299] or generation of monoclonal antibody molecules by continuous cell lines in culture. These include but are not limited to, the hybridoma technique, the human B-cell hybridoma technique, and the Epstein-Bar-Virus (EBV)-hybridoma technique [Kohler G., et al. (1975) Nature 256:495-497, Kozbor D., et al. (1985) J. Immunol. Methods 81:31-42, Cote R. J. et al. (1983) Proc. Natl. Acad. Sci. 80:2026-2030, Cole S. P. et al. (1984) Mol. Cell. Biol. 62:109-120].

In one embodiment, the antibodies are generated by immunizing a subject (e.g. a rodent such as a mouse) with a cyclic peptide that mimics loop 3 of KLK4.

In one embodiment, the immunizing cyclic peptide comprises the amino acid sequence as set forth in SEQ ID NO: 11.

Methods of cyclizing peptides are known in the art, see for instance in WO2010/041237, which is hereby incorporated by reference.

The cyclization may be via N- to C-terminal, N-terminal to side chain and N-terminal to backbone, C-terminal to side chain, C-terminal to backbone, side chain to backbone and side chain to side chain, as well as backbone to backbone cyclization.

Cyclization of the peptide may also take place through non-amino acid organic moieties comprised in the polypeptide.

For example, a peptide according to the teachings of the present invention can include at least two cysteine residues flanking the core peptide sequence (SEQ ID NO: 11). In this case, cyclization can be generated via formation of S—S bonds between the two Cys residues. Side chain to side chain cyclization can also be generated via formation of an interaction bond of the formula -(—CH₂-) n-S—CH₂—C—, wherein n=1 or 2, which is possible, for example, through incorporation of Cys or homoCys and reaction of its free SH group with, e.g., bromoacetylated Lys, Orn, Dab or Dap. Furthermore, cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H-N((CH₂)n-COOH)—C(R)H—COOH or H—N((CH₂)n-COOH)—C(R)H—NH₂, wherein n=1-4, and further wherein R is any natural or non-natural side chain of an amino acid.

Cyclic peptides can be joined together by a peptide bond, a disulfide linkage between two amino acid residues such as cysteine residues, or by any other suitable linking group. Nonpeptidal linking groups can be any chemical moiety that can react with functional groups at each end of the peptide chain to form a link therebetween. For example, two ends of a peptide chain can be linked together by a non-protein amino acid such as 3-aminobutyric acid or by a disulfide formed from nonpeptidal thiol groups such as a thioglycolic amide at the amino terminal end and amide formed from 2-aminoethane thiol at the carboxy terminal end, for example.

Hereinthroughout, the phrases “disulfide bridge” and “disulfide bond” are used interchangeably, and describe a —S—S— bond.

According to another embodiment the cyclization is effected using a coupling agent.

The term “coupling agent”, as used herein, refers to a reagent that can catalyze or form a bond between two or more functional groups intra-molecularly, inter-molecularly or both. Coupling agents are widely used to increase polymeric networks and promote crosslinking between polymeric chains, hence, in the context of some embodiments of the present invention, the coupling agent is such that can promote crosslinking between polymeric chains; or such that can promote crosslinking between amino functional groups and carboxylic functional groups, or between other chemically compatible functional groups of polymeric chains. In some embodiments of the present invention the term “coupling agent” may be replaced with the term “crosslinking agent”. In some embodiments, one of the polymers serves as the coupling agent and acts as a crosslinking polymer.

By “chemically compatible” it is meant that two or more types of functional groups can react with one another so as to form a bond.

According to some embodiments of the present invention, the coupling agent can be selected according to the type of functional groups and the nature of the crosslinking bond that can be formed therebetween. For example, carboxyl coupling directly to an amine can be afforded using a carbodiimide type coupling agent, such as EDC; amines may be coupled to carboxyls, carbonyls and other reactive functional groups by N-hydroxysuccinimide esters (NHS-esters), imidoester, PFP-ester or hydroxymethyl phosphine; sulfhydryls may be coupled to carboxyls, carbonyls, amines and other reactive functional groups by maleimide, haloacetyl (bromo- or iodo-), pyridyldisulfide and vinyl sulfone; aldehydes as in oxidized carbohydrates, may be coupled to other reactive functional groups with hydrazide; and hydroxyl may be coupled to carboxyls, carbonyls, amines and other reactive functional groups with isocyanate.

Hence, suitable coupling agents that can be used in some embodiments of the present invention include, but are not limited to, carbodiimides, NHS-esters, imidoesters, PFP-esters or hydroxymethyl phosphines.

In cases where the immunizing peptides are too small to elicit a strong immunogenic response, such antigens (haptens) can be coupled to antigenically neutral carriers such as keyhole limpet hemocyanin (KLH) or serum albumin [e.g., bovine serum albumin (BSA)] carriers (see U.S. Pat. Nos. 5,189,178 and 5,239,078 and the Examples section). Coupling to carrier can be effected using methods well known in the art; For example, direct coupling to amino groups can be effected and optionally followed by reduction of imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The present invention further contemplates immunization protocols which include subsequent immunization with KLK4 (i.e. boosts) so as to encourage affinity maturation and generate antibodies that have a high affinity to KLK4. Boosting typically is carried out at least two weeks following initial immunization.

The titers of the immune serum can readily be measured using immunoassay procedures which are well known in the art.

The antisera obtained can be used directly or monoclonal antibodies may be obtained as described hereinabove.

Using the above described methods, antibodies may be generated that bind to a cyclic peptide having an amino acid sequence as set forth in SEQ ID NO: 9 with a dissociation constant (KD) of less than 500 nM, less than 50 nM, less than 5 nM as determined by ELISA. In one embodiment, the antibody binds to the amino acid sequence as set forth in SEQ ID NO: 9, with a KD of 10⁸ M or less, e.g., from 10⁸ M to 10¹³ M, e.g., from 10⁹ M to 10¹³ M, as determined by ELISA.

It will be appreciated that antibodies which have further undergone affinity maturation towards human KLK4 may have a dissociation constant with (KD) of less than 500 nM, less than 50 nM, less than 5 nM, e.g., from 10⁸ M to 10¹³ M. e.g., from 10⁹ M to 10¹³ M, for human KLK4 as determined by ELISA.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The term “human KLK4” refers to the serine protease, having a SwissProt No. Q9Y5K2 and a UniProt No. EC: 3.4.21. An exemplary amino acid sequence of KLK4 is set forth in SEQ ID NO: 10.

In one embodiment, the antibody binds with a higher affinity for KLK4 than for at least one additional member of the kallikrein family, such as KLK10 or KLK7, (e.g. at least two fold higher, at least five fold higher or even at least ten fold higher.

The antibodies disclosed herein may be inhibitory antibodies (i.e. KLK4 inhibitory antibodies). According to a particular embodiment, the antibody inhibits human KLK4 protease activity with an IC50 of less than 100 nM, or less than 90 nM, or less than 80 nM, or less than 70 nM, or less than 60 nM, or less than 50 nM, or less than 20 nM, or less than 10 nM, or less than 10 nM.

The antibodies disclosed herein may also affect (e.g. decrease) migration of cancer cells (e.g. ovarian cancer cells) (e.g. as measured using a wound healing assay). Furthermore, the antibodies disclosed herein may decrease proliferation of cancer cells (e.g. ovarian cancer cells).

According to one embodiment, the antibody comprises an antigen recognition domain having complementarity determining region (CDR) amino acid sequences as set forth in: SEQ ID NOs: 1 (CDR1), 2 (CDR2) and 2 (CDR3), sequentially arranged from N to C on a light chain of the antibody) and SEQ ID NOs: 4 (CDR1), 5 (CDR2) and 6 (CDR3), sequentially arranged from N to Con a heavy chain of the antibody.

The light chain of the antibody may comprises an amino acid sequence at least 90%, 91%, 92%, 93%, 94%, 95%, 96%, 97%, 98%, 99% identical to SEQ ID NO: 7.

The heavy chain of the antibody may comprises an amino acid sequence at least 90%, 91%, 92%, 93%, 94%, 95%, 96%, 97%, 98%, 99% identical to SEQ ID NO: 8.

Without being bound to theory, it is contemplated that the presently disclosed antibody binds to an epitope of KLK4 comprising at least one, two, three, four, five or more of the amino acid residues selected from the group consisting of Ser23, Pro24, His25, His71, Ser72, Ala74A, Asp75, Gln76, Glu77, Pro78, Gly79, Ser80, Gln81, Ser113, Glu114, Ser115, Asp116, Thr117, Ile118, and Val154 according to standard protease numbering

The term “epitope” denotes the site on an antigen, either proteinaceous or non-proteinaceous, to which an anti-KLK4 antibody binds. Epitopes can be formed both from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Also contemplate are antibodies that bind to the same epitope as the antibody which has the above disclosed CDR sequences. In addition, additional antibodies are contemplated that bind to the immunizing peptide disclosed herein.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to KLK4 based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Also competitive binding can be used to easily determine whether an antibody binds to the same epitope of KLK4 as, or competes for binding with, an anti-KLK4 antibody.

For example, an “antibody that binds to the same epitope” as a reference anti-KLK4 antibody refers to an antibody that blocks binding of the reference anti-KLK4 antibody, respectively, to its antigen in a competition assay by 50% or more, and conversely, the reference antibody blocks binding of the antibody to its antigen in a competition assay by 50% or more. Also for example, to determine if an antibody binds to the same epitope as a reference anti-KLK4 antibody, the reference antibody is allowed to bind to KLK4 under saturating conditions. After removal of the excess of the reference anti-KLK4 antibody, the ability of an anti-KLK4 antibody in question to bind to KLK4 is assessed. If the anti-KLK4 antibody is able to bind to KLK4 after saturation binding of the reference anti-KLK4 antibody, it can be concluded that the anti-KLK4 antibody in question binds to a different epitope than the reference anti-KLK4 antibody. But, if the anti-KLK4 antibody in question is not able to bind to KLK4 after saturation binding of the reference anti-KLK4 antibody, then the anti-KLK4 antibody in question may bind to the same epitope as the epitope bound by the reference anti-KLK4 antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to KLK4, then it can be concluded that the anti-KLK4 antibody in question and the reference anti-KLK4 antibody compete for binding to KLK4.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

It will be appreciated that for human therapy or diagnostics, humanized antibodies are preferably used. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will include at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147 (1): 86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

As mentioned, the antibody described herein may be a recombinant antibody.

A method of making a recombinant antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. Sec, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described. e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3 A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as YO, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., YO, NSO, Sp20 cell).

As mentioned, the antibody provided herein may also be a multispecific antibody, e.g., a bispecific antibody. “Multi specific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for KLK4 and the other specificity is for any other antigen (e.g. KLK1, KLK13, KLK5, KLK8/KLK11, KLK12/KLK15, KLK6, KLK2, KLK3, KLK14, KLK9, KLK7 or KLK10). In certain aspects, bispecific antibodies may bind to two (or more) different epitopes of an antigen. Multispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)) and “knob-in hole” engineering (see, e.g., U.S. Pat. No. 5,731,168, and Atwell et ah, J. Mol. Biol. 270:26 (1997)). Nonlimiting exemplary knob-in-hole substitutions include T366W (knob) and T366S/L368A/Y407V (hole). In some embodiments, the knob-in-hole substitutions are in IgG1 constant domains.

Multi-specific antibodies may also be made by engineering electrostatic steering effects for making antibody Fc-heterodimeric molecules. See, e.g., WO 2009/089004; Dillon et ah, Mabs 9 (2): 213-230 (2017). As a nonlimiting example, in a bispecific antibody comprising two heavy chain variable regions and two light chain variable regions, a first heavy chain variable region may comprise a Q39E substitution (Kabat numbering) and a first light chain variable region may comprise a Q38K substitution (Kabat numbering); and a second heavy chain variable region may comprise a Q39K substitution (Kabat numbering) and a second light chain variable region may comprise a Q38E substitution (Kabat numbering). In some embodiments, the Q39E/Q38K and Q39K/Q38E substitutions reduce mispairing of the heavy and light chains of the bispecific antibody. Similarly, a first heavy chain constant region may comprise a S183K substitution (EU numbering) and a first light chain constant region may comprise a V133E substitution (EU numbering), and the second heavy chain constant region may comprise a S183E substitution (EU numbering) and a second light chain constant region may comprise a V133K substitution (EU numbering). In some embodiments, the S183K/V133E and S183E/V133K substitutions reduce mispairing of the heavy and light chains of the bispecific antibody.

According to some embodiments of the invention, the antibody may be conjugated to a functional moiety (also referred to as an “immunoconjugate”) such as a detectable or a therapeutic moiety. The immunoconjugate molecule can be an isolated molecule such as a soluble and/or a synthetic molecule.

Various types of detectable or reporter moieties may be conjugated to the antibody of the invention. These include, but not are limited to, a radioactive isotope (such as [125] iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, U K. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the antibody of the invention [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The affinity tag (or a member of a binding pair) can be an antigen identifiable by a corresponding antibody [e.g., digoxigenin (DIG) which is identified by an anti-DIG antibody) or a molecule having a high affinity towards the tag [e.g., streptavidin and biotin]. The antibody or the molecule which binds the affinity tag can be fluorescently labeled or conjugated to enzyme as described above.

Various methods, widely practiced in the art, may be employed to attach a streptavidin or biotin molecule to the antibody of the invention. For example, a biotin molecule may be attached to the antibody of the invention via the recognition sequence of a biotin protein ligase (e.g., BirA) as described in the Examples section which follows and in Denkberg, G. et al., 2000. Eur. J. Immunol. 30:3522-3532. Alternatively, a streptavidin molecule may be attached to an antibody fragment, such as a single chain Fv, essentially as described in Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson).

According to some embodiments of the invention, biotin conjugated antibodies are bound to a streptavidin molecule to form a multivalent composition (e.g., a dimmer or tetramer form of the antibody).

Table 1 provides non-limiting examples of identifiable moieties which can be conjugated to the antibody of the invention.

TABLE 1

| Nucleic Acid sequence | Amino Acid sequence | |
|---|---|---|
| (GenBank | (GenBank | |
| Accession No.) | Accession No.) | Identifiable Moiety |
| AF435427 | AAL33912 | Green Fluorescent |
| | | protein |
| AY042185 | AAK73766 | Alkaline phosphatase |
| A00740 | CAA00083 | Peroxidase |
| Nucleotides 790-807 of | Amino acids 264-269 of | Histidine tag |
| GenBank Accession No. | GenBank Accession No. | |
| AF329457 | AAK09208 | |
| Nucleotides 817-849 of | Amino acids 273-283 of | |
| GenBank Accession No. | GenBank Accession No. | Myc tag |
| AF329457 | AAK09208 | |
| | | Biotin ligase tag |
| AF435432 | AAL33917 | orange fluorescent |
| | | protein |
| EU626139 | ACH42114 | Beta galactosidase |
| AF283893 | AAM49066 | Streptavidin |

As mentioned, the antibody may be conjugated to a therapeutic moiety. The therapeutic moiety can be, for example, a cytotoxic moiety, a toxic moiety, a cytokine moiety and a second antibody moiety comprising a different specificity to the antibodies of the invention.

Non-limiting examples of therapeutic moieties which can be conjugated to the antibody of the invention are provided in Table 2, hereinbelow.

TABLE 2

| Nucleic acid sequence | Amino acid sequence | |
|---|---|---|
| (GenBank | (GenBank | Therapeutic |
| Accession No) | Accession No) | moiety |
| EU090068 | ABU63124 | Pseudomonas |
| | | exotoxin |
| AY820132.1 | AAV70486 | Diphtheria toxin |
| A02159 | CAA00227 | interleukin 2 |
| X03884 | P07766 | CD3 |
| NM_000569.6 | NP_000560.5 | CD16 |
| NM_000589.2 | NP_000580.1 | interleukin 4 |
| K02883 | P01892 | HLA-A2 |
| M57627 | P22301 | interleukin 10 |
| EQ975183 | EEF27734 | Ricin toxin |

The functional moiety (the detectable or therapeutic moiety of the invention) may be attached or conjugated to the antibody of the invention in various ways, depending on the context, application and purpose.

When the functional moiety is a polypeptide, the immunoconjugate may be produced by recombinant means. For example, the nucleic acid sequence encoding a toxin (e.g., PE38KDEL) or a fluorescent protein [e.g., green fluorescent protein (GFP), red fluorescent protein (RFP) or yellow fluorescent protein (YFP)] may be ligated in-frame with the nucleic acid sequence encoding the antibody of the invention and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the antibody of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Inhibitory anti-KLK4 antibodies disclosed herein may be used for treating a subject having a disease associated with an up-regulation of KLK4.

The subject is typically a mammal, e.g. a human.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In one embodiment, the antibody is used to treat an inflammatory skin disease (e.g. by reducing skin inflammatory cytokines, such as IL-8, TNFα, IL-6, IL-4, and G-CSF.

Examples of inflammatory skin diseases include, but are not limited to Netherton Syndrome, atopic dermatitis and psoriasis.

In another embodiment, the antibody is used to treat cancer.

The term “cancer” as used herein refers to an uncontrolled, abnormal growth of a host's own cells which may lead to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s),” “neoplasm(s),” and “tumor(s)” are used herein interchangeably. As used herein, “cancer” refers to all types of cancer or neoplasm or malignant tumors including leukemias, carcinomas and sarcomas, whether new or recurring.

Specific examples of cancers that may be treated using the antibody described herein include, but are not limited to adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; triple negative breast cancer, Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with cosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, malignant melanoma, meningioma; multiple endocrine neoplasia; multiple myeloma, myeloid malignancy, predisposition to; myxosarcoma, neuroblastoma; osteosarcoma; osteocarcinoma, ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, basal cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, and the like.

According to a particular embodiment, the cancer is cancer is selected from the group consisting of prostate, breast and ovarian cancer.

The antibody can be provided to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the multispecific antibody accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (multispecific antibody) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (Sec e.g., Fingl, et al., 1975, in “The Pharmacological Basis of Therapeutics”, Ch. 1 p. 1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

According to a specific embodiment, the dosing of the antibody can be 0.1-100 mg/kg.

According to a specific embodiment, the dosing of the antibody can be 0.1-100 mg/kg. According to a specific embodiment, the dosing of the antibody can be 0.1-80 mg/kg. According to a specific embodiment, the dosing of the antibody can be 0.1-60 mg/kg. According to a specific embodiment, the dosing of the antibody can be 0.1-50 mg/kg. According to a specific embodiment, the dosing of the antibody can be 0.1-40 mg/kg. According to a specific embodiment, the dosing of the antibody can be 0.1-30 mg/kg. According to a specific embodiment, the dosing of the antibody can be 0.1-20 mg/kg. According to a specific embodiment, the dosing of the antibody can be 0.1-10 mg/kg. According to a specific embodiment, the dosing of the antibody can be 1-100 mg/kg. According to a specific embodiment, the dosing of the antibody can be 10-100 mg/kg. According to a specific embodiment, the dosing of the antibody can be 20-100 mg/kg. According to a specific embodiment, the dosing of the antibody can be 30-100 mg/kg. According to a specific embodiment, the dosing of the antibody can be 40-100 mg/kg. According to a specific embodiment, the dosing of the antibody can be 50-100 mg/kg. According to a specific embodiment, the dosing of the antibody can be 60-100 mg/kg. According to a specific embodiment, the dosing of the antibody can be 70-100 mg/kg.

According to a specific embodiment, the dosing of the antibody can be 1-20 mg/kg. According to a specific embodiment, the dosing of the antibody can be 1-15 mg/kg. According to a specific embodiment, the dosing of the antibody can be 1-10 mg/kg. According to a specific embodiment, the dosing of the antibody can be 1-5 mg/kg. According to a specific embodiment, the dosing of the antibody can be 2-20 mg/kg. According to a specific embodiment, the dosing of the antibody can be 4-20 mg/kg. According to a specific embodiment, the dosing of the antibody can be 6-20 mg/kg. According to a specific embodiment, the dosing of the antibody can be 8-20 mg/kg. According to a specific embodiment, the dosing of the antibody can be 10-20 mg/kg. According to a specific embodiment, the dosing of the antibody can be 12-20 mg/kg. According to a specific embodiment, the dosing of the antibody can be 15-20 mg/kg. According to a specific embodiment, the dosing of the antibody can be 18-20 mg/kg. According to a specific embodiment, the dosing of the antibody can be 1-5 mg/kg. According to a specific embodiment, the dosing of the antibody can be 2-10 mg/kg. According to a specific embodiment, the dosing of the antibody can be 5-10 mg/kg.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In one aspect, the anti-KLK4 antibody is for use in a method of diagnosis or detection. In a further aspect, a method of detecting the presence KLK4 in a biological sample is provided. In certain aspects, the method comprises contacting the biological sample with an anti-KLK4 antibody as described herein under conditions permissive for binding of the antibody to its antigen, and detecting whether a complex is formed between the antibody and the antigen. Such method may be an in vitro or in vivo method. In some embodiments, methods of selecting patients for treatment with an antibody provided herein comprise determining KLK4 expression in a sample from the patient. For diagnosis or detection, it is contemplated that the antibody is labeled with a detectable moiety, examples of which are provided herein above.

It is expected that during the life of a patent maturing from this application many relevant agonistic KLK4 antibodies will be developed and the scope of the term anti-KLK4 antibody is intended to include all such new technologies a priori.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Immunization protocol: Five balb-c mice were injected subcutaneous with KLK4 and Loop-KLK4-KLH (CGLHSLEADQEPGSCC-SEQ ID NO: 9) using adjuvant CFA. In detail, boosts with antigens KLK4 (amino acid sequence as set forth in SEQ ID NO: 10) and Loop-KLK4-KLH was performed every 2-3 weeks:
3 injections with loop-KLK-4-KLH-35 μg/mouse;
Bleeding: Elisa test against antigen loop-KLK4-BSA;
2 injections with KLK-4-35 μg/mouse;
Bleeding: Elisa test against both antigens loop-KLK4-BSA and KLK-4;
1 injection with Loop-KLK-4-KLH-35 μg/mouse;
Bleeding: Elisa test against both antigens loop-KLK4-BSA and KLK-4;
3 additional injections with both antigens; and
Bleeding: Elisa test against both antigens loop-KLK4-BSA and KLK-4.

Following this protocol, spleen and lymph nodes was harvested for generation of hybridomas. ELISA was used for selecting the best clones against both antigens. Following the cloning, subcloning was performed and the best clone was sequenced.

ELISA binding assay: A ninety-six-well plate (Nunc) was coated with KLK4 and Loop-KLK4 at 10 g/ml. After blocking with 2% BSA in PBS, the plate was incubated with the antibodies for 1 h at 37° C. Bound antibodies were detected by peroxidase-conjugated antibody goat anti-human (Jackson ImmunoResearch). EC₅₀ was calculated with GraphPad Prism from Find ECanything curve fitting analysis.

Fibrinogen proteolysis assay: Increasing concentrations of antibodies (anti-KLK4) (500 nM, 250 nM, 125 nM, 62.5 nM) were incubated with protease (final KLK4 concentration=2.5 nM) for 20 min in 200 μl assay buffer (100 mM Tris-HCl, 100 mM NaCl2, 0.005% triton-X, pH 8.0). Enzyme activity was initiated by addition of 7 uM fibrinogen substrate in 100 μl assay buffer (final concentration 100 M). Proteolysis proceeded for 90 min before termination by boiling in SDS-PAGE sample buffer. Proteolysis fragments were separated on 10% polyacrylamide gels. Assay buffer: 100 mM Tris-HCl, 100 mM NaCl₂, 0.005% triton-X, pH 8.0.

In vitro wound healing assay: ES2 and OVCAR3 confluent cell layers were wounded by scratching with a sterile 10 μL pipette tip. Detached cells were removed by washing two times with PBS and fresh culture medium was added in the absence or presence of conditioned media. The wound closure was monitored, at 0, 6, 12, 24, 36 h for ES2 cells and at 0, 24, 48 h for the OVCAR3 cells, using a digital camera connected to a microscope. Wound surface area was quantified by image analysis (ImageJ2, Fiji v2.3.0/1.53f).

Molecular Modelling: A model of the Fv portion of the antibody was constructed using RosettaAntibody software. 10 homology models were initially constructed, followed by exhaustive modelling of CDR H3, resulting in 2900 models. Top 10 models, scored by energy, were selected for docking. 1000 docking calculations was performed using the top 10 H3 models, and results were ranked according to interface energy, visual inspection, and interface shape complementarity (Sc).

### Results

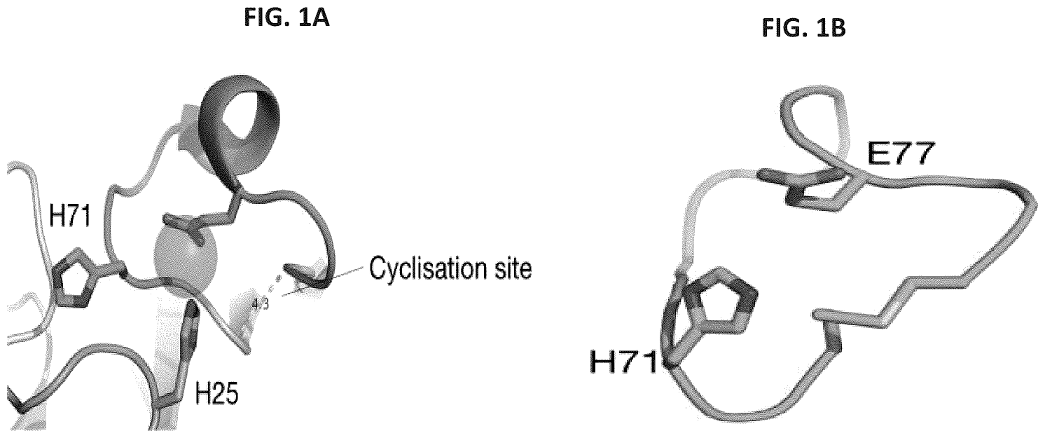

Mice were injected with the KLK4 loop 3 peptide (FIGS. 1A-B) alongside an adjuvant and were monitored for an initiated immune response. Full KLK4 was injected in order to mature the antibodies against the complete antigen structure. This resulted in the generation of antibodies that bind to KLK4 in 8 to 18 weeks from initial injection of adjuvants.

A monoclonal antibody was generated and sequenced as follows.

IgK(VL):.
(SEQ ID NO: 7)
IgG1(VH):.
(SEQ ID NO: 8)

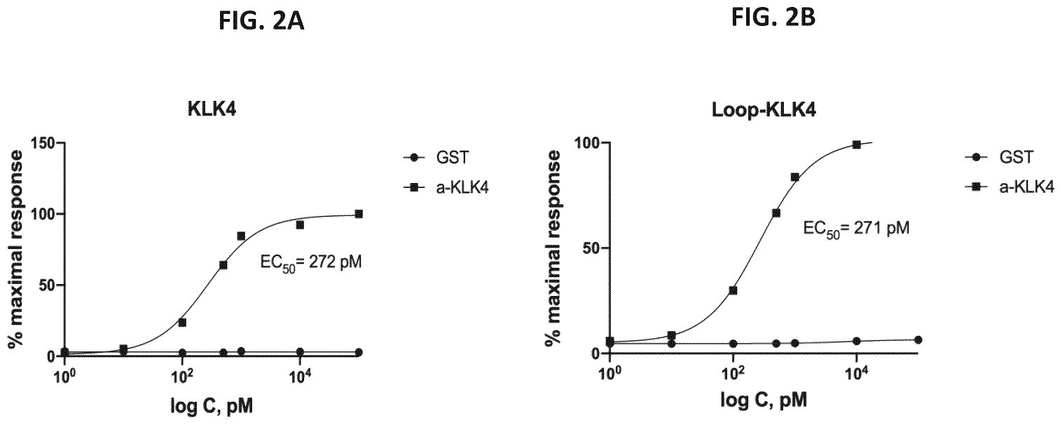

Interaction of KLK4 monoclonal antibody was tested by ELISA using KLK4 & KLK4-Loop3. As a control, anti-GST monoclonal antibody was used. EC50 against KLK4 was measured at 272 pM and against Loop3-KLK4 at 271 pM (FIGS. 2A-B).

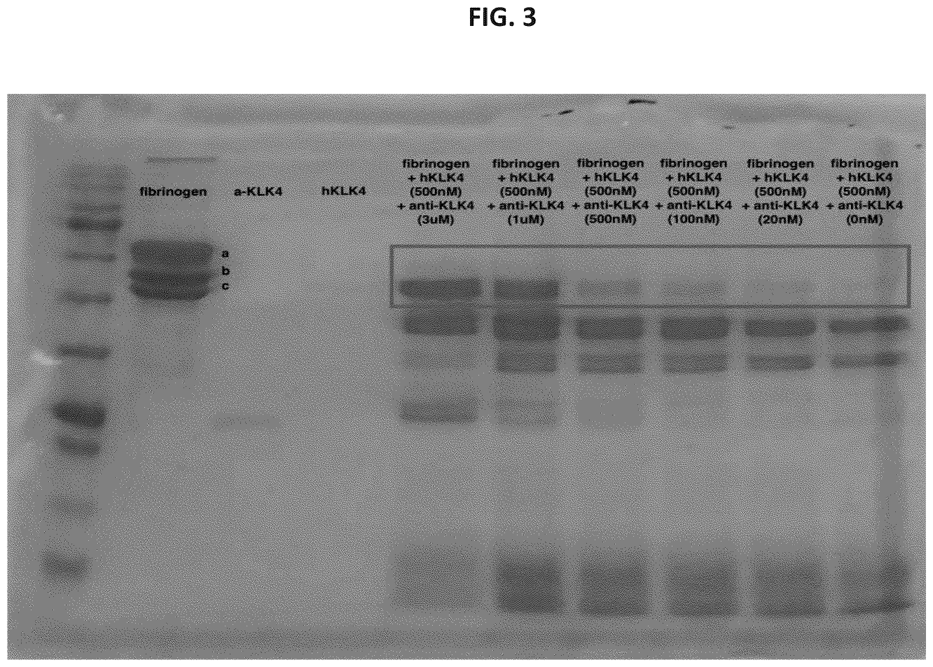

The ability of anti-KLK4 antibody to inhibit KLK4 proteolysis of fibrinogen, (a known substrate for the enzyme) was tested. Anti-KLK4 was found to be a potent inhibitor of fibrinogen proteolysis by KLK4, with almost complete inhibition at 20 nM (FIG. 3).

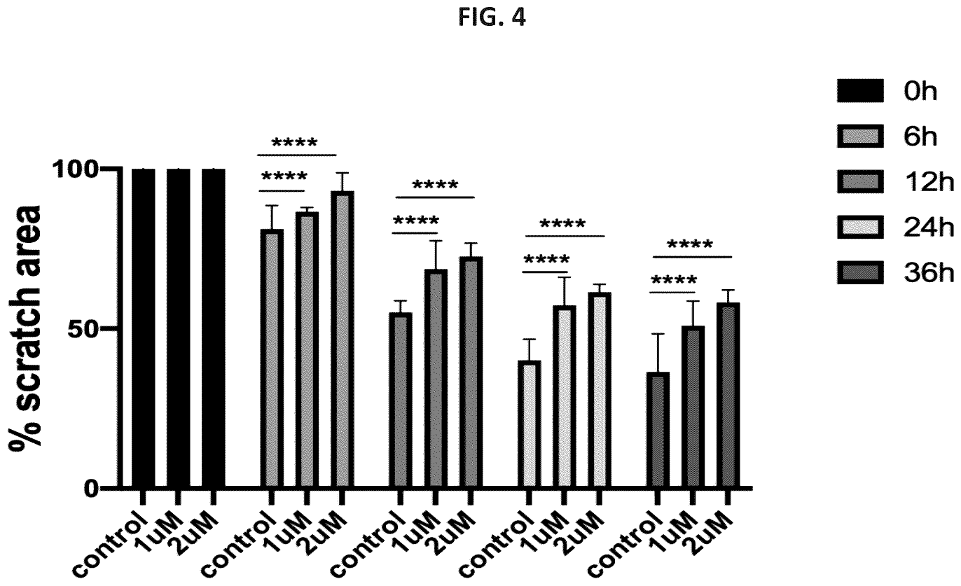

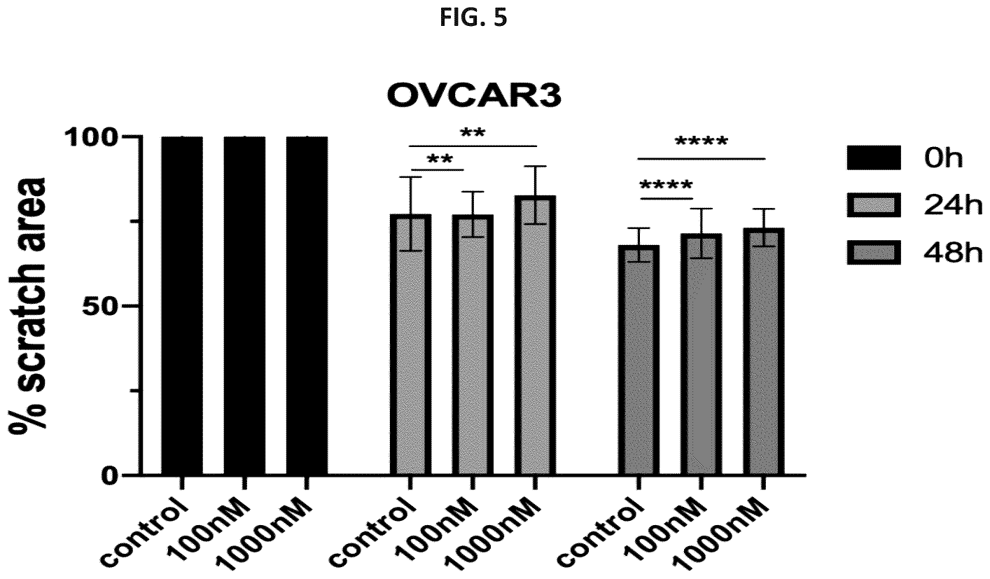

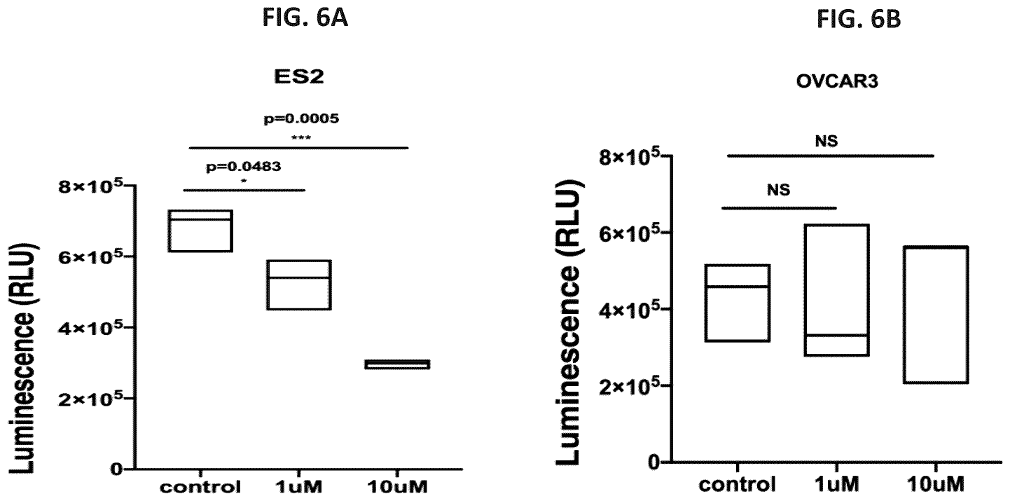

Further, evaluation of the anti-KLK4 antibody was performed in two ovarian carcinoma cell lines ES2 and OVCAR3. ES-2 is a fibroblast-like cell line which has high proliferation rate. OVCAR3 cells have an epithelial phenotype. Both were isolated from the malignant ascites of a patient with progressive adenocarcinoma of the ovary. Initially, the ability of anti-KLK4 to affect migration of these cells was analyzed using a wound healing assay. Anti-KLK4 antibody (at two different concentrations (1 μM and 2 μM)) inhibited migration of ES2 cells (FIG. 4). In addition, anti-KLK4 antibody (100 nM) inhibited migration of OVCAR3 cells (FIG. 5). Anti-KLK4 antibody was found to affect proliferation of ES2 cells (FIGS. 6A-B). All these data highlight that the new antibody against KLK4 successfully inhibits the activity of KLK4 and also reduces the metastatic potential of two ovarian cell line with different metastatic characteristics.

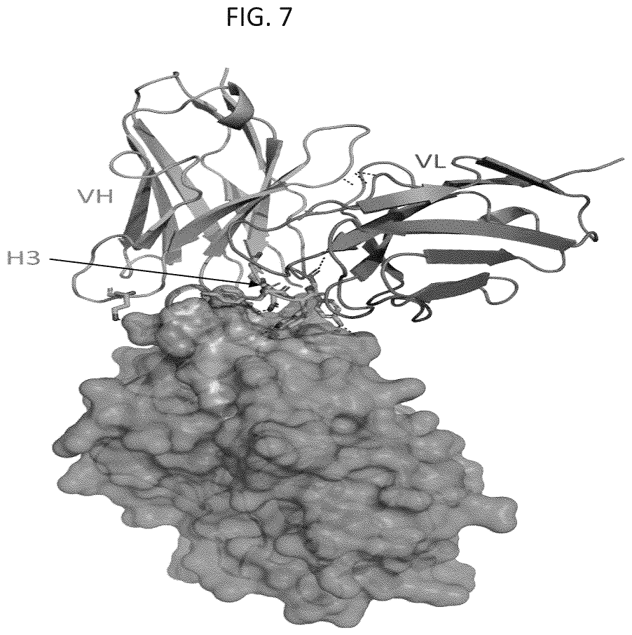

A molecular model of the antibody was built and docking experiments were carried out in order to generate a model of the KLK4-Ab complex (FIG. 7). V_{H} dominates the KLK4-Ab interaction, with H3 interacting with loop 3.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

What is claimed is:

1. An isolated antibody that binds to a cyclized peptide having an amino acid sequence as set forth in SEQ ID NO: 9 with an EC50 of less than 500 nM, as determined by ELISA.

2. The isolated antibody of claim 1, comprising an antigen recognition domain having complementarity determining region (CDR) amino acid sequences as set forth in: SEQ ID NOs: 1 (CDR1), 2 (CDR2) and 2 (CDR3), sequentially arranged from N to C on a light chain of the antibody) and SEQ ID NOs: 4 (CDR1), 5 (CDR2) and 6 (CDR3), sequentially arranged from N to C on a heavy chain of the antibody.

3. The isolated antibody of claim 1, being capable of:
binding to human KLK4 with an EC50 of less than 500 nM, as measured by ELISA;
binding the human KLK4 with a higher affinity as compared to human KLK10, as measured by ELISA; and/or
inhibiting KLK4 protease activity with an IC50 of less than 100 nM.

4. The isolated antibody of claim 3, wherein the light chain comprises an amino acid sequence at least 90% identical to SEQ ID NO: 7 and/or the heavy chain comprises an amino acid sequence at least 90% identical to SEQ ID NO: 8.

5. An isolated antibody which binds specifically to human KLK4, comprising an antigen recognition domain having complementarity determining region (CDR) amino acid sequences as set forth in: SEQ ID NOs: 1 (CDR1), 2 (CDR2) and 2 (CDR3), sequentially arranged from N to C on a light chain of the antibody) and SEQ ID NOs: 4 (CDR1), 5 (CDR2) and 6 (CDR3), sequentially arranged from N to C on a heavy chain of the antibody.

6. An isolated antibody that competes for binding with the antibody of claim 5.

7. An isolated antibody that binds to the same epitope as the antibody of claim 5.

8. A method of producing an antibody comprising culturing a host cell comprising a nucleic acid encoding the antibody of claim 1 so that the antibody is produced.

9. An immunoconjugate comprising the antibody of claim 1.

10. A cyclized peptide comprising the amino acid sequence as set forth in SEQ ID NO: 11.

11. The cyclized peptide of claim 10, consisting of the amino acid sequence as set forth in SEQ ID NO: 9.

12. The cyclized peptide of claim 10, being coupled to an antigenically neutral carrier optionally wherein the antigenically neutral carrier comprises keyhole limpet hemocyanin (KLH) or serum albumin.

13. A pharmaceutical composition comprising the antibody of claim 1 and a pharmaceutically acceptable carrier.

14. A method of treating a disease associated with an up-regulation of KLK4 in a subject in need thereof comprising administering to the subject a therapeutically effective amount of the antibody of claim 1, thereby treating the disease.

15. The method of claim 14, wherein the disease is cancer.

16. The method of claim 15, wherein the cancer is selected from the group consisting of prostate, breast and ovarian cancer.

17. The method of claim 14, wherein the disease is an inflammatory skin disease.

18. The method of claim 17, wherein the inflammatory skin disease is selected from the group consisting of Netherton Syndrome, atopic dermatitis and psoriasis.

19. Am ex vivo method of inhibiting a biological activity of KLK4 comprising contacting cells expressing KLK4 with an effective amount of the antibody of claim 1, thereby inhibiting the biological activity of KLK4.

20. A method of screening for an antibody that binds KLK4 comprising contacting a candidate antibody with the cyclized peptide of claim 10, wherein a binding of said candidate antibody to said cyclized peptide with an affinity above a predetermined threshold is indicative of an antibody that binds to KLK4.


SEQUENCE LISTING (11 sequences)

SEQ ID NO: 1 - protein, 12 aa, synthetic construct
  REGION 1..12: note = Complementarity-determining region (CDR)
RASSSVSSTYLH

SEQ ID NO: 2 - protein, 7 aa, synthetic construct
  REGION 1..7: note = Complementarity-determining region (CDR)
STSNLAS

SEQ ID NO: 3 - protein, 9 aa, synthetic construct
  REGION 1..9: note = Complementarity-determining region (CDR)
QQYSSYPLT

SEQ ID NO: 4 - protein, 7 aa, synthetic construct
  REGION 1..7: note = Complementarity-determining region (CDR)
GFTFKTY

SEQ ID NO: 5 - protein, 8 aa, synthetic construct
  REGION 1..8: note = Complementarity-determining region (CDR)
RNKNNNYA

SEQ ID NO: 6 - protein, 8 aa, synthetic construct
  REGION 1..8: note = Complementarity-determining region (CDR)
VNYWYFDV

SEQ ID NO: 7 - protein, 108 aa, synthetic construct
  REGION 1..108: note = monoclonal antibody IgK(VL)
EIVMTQSTAIMSASPGEKVTMTCRASSSVSSTYLHWYQQKSGASPKLWIYSTSNLASGVPARFSGSGSGTSYSLTVSSVEAEDAATYYCQQYSSYPLTFGGGTKLEIK

SEQ ID NO: 8 - protein, 119 aa, synthetic construct
  REGION 1..119: note = monoclonal antibody IgG1(VH)
EVQLVESGGGLVQPKGSLKLSCAASGFTFKTYAMTWVRQTPGKGLEWVARIRNKNNNYATFYADSVKDRFTISRDDSQSMLYLLMNNLKTEDTAIYYCVRVNYWYFDVWGAGTTVTVSS

SEQ ID NO: 9 - protein, 16 aa, synthetic construct
  REGION 1..16: note = cyclized peptide
CGLHSLEADQEPGSCC

SEQ ID NO: 10 - protein, 223 aa, synthetic construct
  REGION 1..223: note = amino acid sequence of KLK4
IINGEDCSPHSQPWQAALVMENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVQA

SEQ ID NO: 11 - protein, 14 aa, synthetic construct
  REGION 1..14: note = immunizing cyclic peptide
GLHSLEADQEPGSC